(19) United States
(12) Patent Application Publication
(10) Pub. No.: US 2023/0085299 A1
(43) Pub. Date: Mar. 16, 2023

(54) TREATMENT APPARATUS AND TREATMENT METHOD

(71) Applicant: TERUMO KABUSHIKI KAISHA, Tokyo (JP)

(72) Inventors: Mayu HATA, Yokohama-shi (JP); Satoru SUEHARA, Ashigarakami-gun (JP); Daiki TOHMA, Fujisawa-shi (JP)

(73) Assignee: TERUMO KABUSHIKI KAISHA, Tokyo (JP)

(21) Appl. No.: 17/895,199

(22) Filed: Aug. 25, 2022

(30) Foreign Application Priority Data
Sep. 13, 2021 (JP) 2021-148298

(51) Int. Cl. A61N 5/06

(52) U.S. Cl. CPC A61N 5/062; A61N 2005/0612; A61N 2005/063; A61N 2005/0666; A61N 2005/0628; A61N 2005/0611

(57) ABSTRACT

A treatment apparatus and a treatment method capable of minimally invasively specifying a lesion extent and checking progress of treatment while performing the treatment for destroying tumor cells. The treatment apparatus that detects and destroys a tumor cell by irradiating an antibody-photosensitive substance adsorbed on a tumor cell membrane with excitation light includes: an elongated tubular shaft having optical transparency; a light irradiation unit configured to emit the excitation light of the antibody-photosensitive substance from an inside of the shaft in a side direction perpendicular to an axial direction of the shaft; and a side direction fluorescence detection unit configured to detect, from the side direction perpendicular to the axial direction of the shaft, fluorescence emitted by the excited antibody-photosensitive substance, and movable in the axial direction with respect to the shaft.

### CROSS-REFERENCES TO RELATED APPLICATIONS

This application claims priority to Japanese Application No. 2021-148298 filed on Sep. 13, 2021, the entire content of which is incorporated herein by reference.

### TECHNOLOGICAL FIELD

The present disclosure relates to a treatment apparatus and a treatment method for cervical cancer.

### BACKGROUND DISCUSSION

As local treatment for cancer, a treatment method using a photoreactive substance is known (for example, see Japanese Patent No. 4,185,313). In particular, in a treatment method using an antibody-photosensitive substance (hydrophilic phthalocyanine), it is expected that, target cells can be specifically destroyed without destroying non-target cells such as normal cells by irradiating the antibody-photosensitive substance accumulated in a tumor with excitation light (for example, near-infrared rays), and a relatively high treatment effect can be achieved while reducing side effects.

In treatment for cervical cancer, as a pre-diagnosis, a cytological diagnosis for diagnosing, under a microscope, cells collected by wiping with a brush or spatula, and a culposcope diagnosis using a culposcope that can be inserted from the vagina to precisely observe a uterine ostium are performed. After the pre-diagnosis, conization is performed, and a definitive diagnosis (a pathological diagnosis) using the excised section can be performed. In the pre-diagnosis, an extent and a severity of a lesion cannot be accurately grasped, and thus the definitive diagnosis is required. By the definitive diagnosis, an advanced stage and a treatment method of cancer can be determined. If the result of the diagnosis on a stump of the conical excised section is negative, the conical excision serves as treatment, and the treatment ends.

In the treatment method using the photoreactive substance, since the cells are not excised, the definitive diagnosis cannot be performed. Therefore, it is not possible to specify a lesion extent before treatment, or to check whether all lesions are treated after the treatment or whether additional treatment is required.

Japanese Patent No. 4,185,313 describes a method of minimally invasively specifying, by irradiating a biological tissue with excitation light (ultraviolet light), a tumor that emits fluorescence different from fluorescence emitted by a normal biological tissue.

### SUMMARY

A treatment apparatus and a treatment method are disclosed, which are capable of minimally invasively specifying a lesion extent and checking progress of treatment while performing the treatment for destroying tumor cells.

A treatment apparatus according to the disclosure can be a treatment apparatus that detects and destroys a tumor cell by irradiating an antibody-photosensitive substance adsorbed on a tumor cell membrane with excitation light, the treatment apparatus including: an elongated tubular shaft having optical transparency; a light irradiation unit configured to emit the excitation light of the antibody-photosensitive substance from an inside of the shaft to a side direction perpendicular to an axial direction of the shaft; and a side direction fluorescence detection unit configured to detect, from the side direction perpendicular to the axial direction of the shaft, fluorescence emitted by the excited antibody-photosensitive substance, and movable in the axial direction with respect to the shaft.

According to the treatment apparatus described above, since the side direction fluorescence detection unit is movable in the axial direction, the fluorescence emitted by the antibody-photosensitive substance excited by the excitation light from the light irradiation unit can be detected by the side direction fluorescence detection unit at a plurality of positions in the axial direction. Therefore, according to this treatment apparatus, it is possible to minimally invasively specify a lesion extent and check progress of treatment while performing the treatment for destroying the tumor cells.

The treatment apparatus may include a puncture portion disposed at a distal end of the shaft. Accordingly, even when there is a lesion at a position deep from a tissue surface, by puncturing a tissue and inserting the shaft into the tissue, it is possible to treat the lesion, specify the lesion extent, and check the progress of the treatment.

The treatment apparatus may include: a light adjustment unit configured to change a direction of the excitation light emitted from the light irradiation unit; and a distal direction fluorescence detection unit configured to detect the fluorescence from a distal direction of the shaft. The shaft may include a base shaft having a distal portion at which the light adjustment unit is disposed, and a distal shaft protruding from the light adjustment unit in the distal direction and having optical transparency, and the light irradiation unit may be movable between an inside of the distal shaft and an inside of the light adjustment unit. Accordingly, by disposing the light irradiation unit inside the distal shaft, it is possible to detect the fluorescence from the side direction by the side direction fluorescence detection unit while emitting the excitation light in the side direction. Further, by disposing the light irradiation unit inside the light adjustment unit, it is possible to detect the fluorescence from the distal direction by the distal direction fluorescence detection unit while emitting the excitation light in the distal direction. Therefore, a tumor in a cervix can be effectively treated by emitting the excitation light from an inside of a cervical canal by disposing the light irradiation unit inside the distal shaft inserted into the cervical canal, and the tumor in the cervix can be effectively treated by emitting the excitation light from a vagina by disposing the light irradiation unit inside the light adjustment unit that is disposed at a site close to the uterine vagina in the vagina.

The side direction fluorescence detection unit may include a ring-shaped scatterer disposed along a peripheral direction of the light irradiation unit and movable in the axial direction along an outer peripheral surface of the light irradiation unit, and an optical waveguide disposed on a proximal side of the scatterer. Accordingly, since the side direction fluorescence detection unit can detect the fluorescence at the plurality of positions in the axial direction, the lesion extent in the axial direction in which the tumor cells are present can be specified.

The side direction fluorescence detection unit may include a plurality of optical fibers, and a plurality of incident portions of the optical fibers may be disposed along a peripheral direction of the light irradiation unit, and are movable in the axial direction along an outer peripheral surface of the light irradiation unit. Accordingly, since the side direction fluorescence detection unit can individually detect the fluorescence at a plurality of positions in the peripheral direction, the lesion extent in the peripheral direction in which the tumor cells are present can be specified.

A treatment apparatus according to another aspect of the disclosure is a treatment apparatus that detects and destroys a tumor cell by irradiating an antibody-photosensitive substance adsorbed on a tumor cell membrane with excitation light, the treatment apparatus including: a light adjustment unit configured to change a direction of light emitted from a light irradiation unit; a base shaft having a distal portion at which the light adjustment unit is disposed; the light irradiation unit configured to emit the excitation light of the antibody-photosensitive substance through the light adjustment unit; and a fluorescence detection unit configured to detect fluorescence emitted by the excited antibody-photosensitive substance and movable in a peripheral direction centered on an axis substantially parallel to an axial center of the base shaft.

According to the treatment apparatus of the another aspect described above, since the fluorescence detection unit is movable in a rotation direction, the fluorescence emitted by the antibody-photosensitive substance excited by the excitation light emitted from the light irradiation unit can be detected by the fluorescence detection unit at the plurality of positions in the peripheral direction. Therefore, according to this treatment apparatus, it is possible to minimally invasively specify the lesion extent and check the progress of the treatment while performing the treatment for destroying the tumor cells.

A treatment method according to the disclosure is a treatment method for detecting and destroying a tumor cell by irradiating an antibody-photosensitive substance adsorbed on a tumor cell membrane of a cervix with excitation light, the treatment method including: administering the antibody-photosensitive substance into a body; after the administration, widening a vagina by a vaginal speculum, and inserting a treatment apparatus into the vagina, the treatment apparatus including an elongated tubular shaft having optical transparency, a light irradiation unit configured to emit the excitation light of the antibody-photosensitive substance, and a side direction fluorescence detection unit configured to detect, from a side direction perpendicular to an axial direction of the shaft, fluorescence emitted by the excited antibody-photosensitive substance; inserting the shaft into a cervical canal while visually checking a distal portion of the shaft and an external uterine ostium; emitting the excitation light by the light irradiation unit from an inside of the shaft in the side direction; and while emitting the excitation light, detecting the fluorescence emitted by the antibody-photosensitive substance by moving the side direction fluorescence detection unit in the axial direction with respect to the shaft, and checking disappearance or reduction of the fluorescence.

According to the treatment method described above, since the fluorescence is detected by moving the side direction fluorescence detection unit in the axial direction while emitting the excitation light from the inside of the cervical canal, it is possible to minimally invasively specify a position of the lesion extent along the axial direction and check the progress of the treatment while performing the treatment for destroying the tumor cells of the cervix.

In the treatment method, moving the light irradiation unit in the axial direction, and while emitting the excitation light by the light irradiation unit from the inside of the shaft in the side direction, detecting the fluorescence emitted by the antibody-photosensitive substance by moving the side direction fluorescence detection unit in the axial direction with respect to the shaft, and checking the disappearance or reduction of the fluorescence may be repeated one or more times after checking the disappearance or reduction of the fluorescence. Accordingly, this treatment method makes it possible to minimally invasively treat the tumor in a wide range along the axial direction of the shaft, specify the lesion extent, and check the progress of the treatment.

A treatment method according to another aspect of the disclosure is a treatment method for detecting and destroying a tumor cell by irradiating an antibody-photosensitive substance adsorbed on a cell membrane of a tumor developed in at least a part in a range from a cervix to a vagina with excitation light, the treatment method including: administering the antibody-photosensitive substance into a body; after the administration, widening the vagina by a vaginal speculum and inserting a treatment apparatus into the vagina, the treatment apparatus including an elongated base shaft, a light adjustment unit disposed at a distal portion of the base shaft and configured to change a direction of light, an elongated tubular distal shaft protruding from the light adjustment unit in a distal direction and having optical transparency, a light irradiation unit configured to emit the excitation light of the antibody-photosensitive substance, a side direction fluorescence detection unit configured to detect, from a side direction perpendicular to an axial direction of the distal shaft, fluorescence emitted by the excited antibody-photosensitive substance, and a distal direction fluorescence detection unit configured to detect the fluorescence from a distal side; inserting the distal shaft into a cervical canal while visually checking the distal shaft; inflating the light adjustment unit in accordance with a shape of an organ; emitting the excitation light by the light irradiation unit in the side direction from an inside of the distal shaft; while emitting the excitation light, detecting the fluorescence emitted by the antibody-photosensitive substance by moving the side direction fluorescence detection unit in the axial direction with respect to the distal shaft, and checking disappearance or reduction of the fluorescence; moving the light irradiation unit to a position where the light adjustment unit is disposed in the axial direction; and while emitting the excitation light through the light adjustment unit, detecting the fluorescence emitted by the antibody-photosensitive substance by the distal direction fluorescence detection unit, and checking the disappearance or reduction of the fluorescence.

According to the treatment method of the another aspect described above, by emitting the excitation light from the inside of the cervical canal and detecting the fluorescence by the side direction fluorescence detection unit, it is possible to minimally invasively treat the tumor in the cervix, specify the position of the lesion extent, and check the progress of the treatment without excising a tissue. Further, by emitting the excitation light from the inside of the light adjustment unit disposed at the site close to the uterine vagina in the vagina and detecting the fluorescence by the distal direction fluorescence detection unit, it is possible to minimally invasively treat the tumor distributed in the uterine vagina, specify the lesion extent, and check the progress of the treatment.

A treatment method according to still another aspect of the disclosure is a treatment method for detecting and destroying a tumor cell by irradiating an antibody-photosensitive substance adsorbed on a cell membrane of a tumor developed in at least a part in a range from a cervix to a vagina, the treatment method including: administering the antibody-photosensitive substance into a body; after the administration, widening the vagina by a vaginal speculum, and inserting a treatment apparatus into the vagina, the treatment apparatus including an elongated base shaft, a light adjustment unit disposed at a distal portion of the base shaft and configured to change a direction of light, a light irradiation unit configured to emit the excitation light of the antibody-photosensitive substance, and a fluorescence detection unit configured to detect fluorescence emitted by the excited antibody-photosensitive substance; inflating the light adjustment unit in accordance with a shape of an organ; and while emitting the excitation light by the light irradiation unit from an inside of the light adjustment unit, detecting the fluorescence emitted by the antibody-photosensitive substance by moving the fluorescence detection unit in a rotation direction centered on an axis substantially parallel to an axial center of the base shaft, and checking disappearance or reduction of the fluorescence.

According to the treatment method of the still another aspect described above, by emitting the excitation light from the inside of the light adjustment unit disposed at the site close to the uterine vagina in the vagina and detecting the fluorescence by the fluorescence detection unit, it is possible to minimally invasively treat the tumor distributed in the uterine vagina, specify the lesion extent, and check the progress of the treatment.

A treatment method according to yet another aspect of the disclosure is a treatment method for detecting and destroying a tumor cell by irradiating an antibody-photosensitive substance adsorbed on a tumor cell membrane with excitation light, the treatment method including: administering the antibody-photosensitive substance into a body; after the administration, forming a hole in a living body by a puncture portion and inserting a distal portion of a treatment apparatus into the living body while checking the distal portion of the treatment apparatus by visual observation or based on an image obtained by an image detecting device such as an ultrasonic diagnostic device, CT or MRI, the treatment apparatus including an elongated tubular shaft having optical transparency, the puncture portion disposed at a distal end of the shaft, a light irradiation unit configured to emit the excitation light of the antibody-photosensitive substance, and a side direction fluorescence detection unit configured to detect, from a side direction perpendicular to an axial direction of the shaft, fluorescence emitted by the excited antibody-photosensitive substance; emitting the excitation light by the light irradiation unit from an inside of the shaft in the side direction; and while emitting the excitation light, detecting the fluorescence emitted by the antibody-photosensitive substance by moving the side direction fluorescence detection unit in the axial direction with respect to the shaft, and checking disappearance or reduction of the fluorescence.

According to the treatment method of the yet another aspect described above, when the lesion is at a position deep from the tissue surface, it is possible to puncture the tissue to cause the treatment apparatus to reach the vicinity of the tumor cells, and detect the fluorescence by moving the side direction fluorescence detection unit in the axial direction while emitting the excitation light from the inside of the punctured tissue. Therefore, it is possible to specify the position of the lesion extent along the axial direction and check the progress of the treatment while performing the treatment for destroying the tumor cells by the excitation light.

A treatment method according to another aspect for detecting and destroying a tumor cell by irradiating an antibody-photosensitive substance adsorbed on a tumor cell membrane of a cervix with excitation light includes: administering the antibody-photosensitive substance into a living body; widening a vagina by a vaginal speculum, and inserting a treatment apparatus into the vagina; inserting of the treatment apparatus into a cervical canal while visually checking a distal portion of a tubular shaft of the treatment apparatus and an external uterine ostium; emitting an excitation light from the treatment apparatus from an inside of the tubular shaft in a side direction; and detecting fluorescence emitted by the antibody-photosensitive substance by moving the treatment apparatus in the axial direction with respect to the tubular shaft, and checking disappearance or reduction of the fluorescence.

### BRIEF DESCRIPTION OF THE DRAWINGS

FIG. 1 is a plan view illustrating a treatment apparatus according to a first embodiment.

FIGS. 2A and 2B are diagrams illustrating a distal portion of the treatment apparatus according to the first embodiment, in which FIG. 2A is a cross-sectional view, and FIG. 2B is a cross-sectional view taken along a line A-A of FIG. 2A.

FIGS. 3A and 3B are diagrams illustrating a side direction fluorescence detection unit according to a modification, in which FIG. 3A is a perspective view illustrating a distal portion of the side direction fluorescence detection unit, and FIG. 3B is a perspective view illustrating one of optical fibers constituting the side direction fluorescence detection unit.

FIG. 4 is a perspective view illustrating a side direction fluorescence detection unit according to another modification.

FIG. 5 is a schematic view illustrating a state in which the treatment apparatus is inserted into a vagina and a cervix.

FIGS. 6A, 6B, and 6C are cross-sectional views illustrating states in which treatment is performed by the treatment apparatus according to the first embodiment, in which FIG. 6A illustrates a state in which a detection unit is positioned at a distal portion of a lumen of a shaft, FIG. 6B illustrates a state in which the detection unit is moving in an axial direction, and FIG. 6C illustrates a state in which a light emitting unit is moved in a proximal direction.

FIG. 7 is a cross-sectional view illustrating a state in which the treatment is performed by using the side direction fluorescence detection unit according to the modification.

FIG. 8 is a plan view illustrating a side direction fluorescence detection unit according to still another modification.

FIG. 9 is a cross-sectional view illustrating a distal portion of a treatment apparatus according to a second embodiment.

FIG. 10 is a cross-sectional view illustrating a state in which treatment is performed by disposing a light emitting unit of the treatment apparatus according to the second embodiment on a distal shaft.

FIG. 11 is a cross-sectional view illustrating a state in which the treatment is performed by disposing the light emitting unit of the treatment apparatus according to the second embodiment in a light adjustment unit.

FIG. 12 is a cross-sectional view illustrating a treatment apparatus according to a modification of the second embodiment.

FIG. 13 is a cross-sectional view illustrating a distal portion of a treatment apparatus according to a third embodiment.

FIGS. 14A and 14B are cross-sectional views illustrating states in which treatment is performed by a treatment apparatus according to a fourth embodiment, in which FIG. 14A illustrates a state in which a detection unit is positioned at a distal portion of a lumen of a shaft, and FIG. 14B illustrates a state in which the detection unit is moving in an axial direction.

### DETAILED DESCRIPTION

Set forth below with reference to the accompanying drawings is a detailed description of embodiments of a treatment apparatus and a treatment method for cervical cancer. Note that since embodiments described below are preferred specific examples of the present disclosure, although various technically preferable limitations are given, the scope of the present disclosure is not limited to the embodiments unless otherwise specified in the following descriptions. Note that for convenience of explanation, dimensions in the drawings may be exaggerated and may be different from actual dimensions. In the present specification and the drawings, components having substantially the same functional configuration are designated by the same reference numerals, and a duplicate description of the components having substantially the same functional configuration will be omitted. In the present specification, a side of a device to be inserted into a body lumen is referred to as a “distal side”, and a side to be operated is referred to as a “proximal side”.

### First Embodiment

A treatment apparatus 10 according to a first embodiment is used for a treatment method for a tumor in a cervix. The treatment apparatus 10 and the treatment method can also be used to simultaneously treat both the tumor in the cervix and a tumor in a vagina. The treatment apparatus 10 and the treatment method can be used for photoimmunotherapy of destroying target cells by irradiating an antibody-photosensitive substance adsorbed on cell membranes of the target cells with near-infrared rays, which serve as excitation light of the antibody-photosensitive substance. The target cells are tumor cells such as cancer cells or precancerous lesion cells. In this treatment method, the antibody-photosensitive substance, which is obtained by absorbing an antibody that specifically binds to only a specific antigen on surfaces of the tumor cells and a photosensitive substance paired with the antibody, can be used as a drug. The antibody is not particularly limited, and can include, for example, panitumbab, trastuzumab, HuJ591, pertuzumab, lapatinib, palbociclib, olaparib, and the like. The photosensitive substance can be, for example, hydrophilic phthalocyanine which is a substance that reacts with near-infrared rays having a wavelength of about 700 nm (IR700), but is not limited to hydrophilic phthalocyanine. When IR700 receives near-infrared rays having a wavelength of about 660 nm to 740 nm, a ligand of a functional group that secures water solubility is broken, causing a structural change from water-soluble to hydrophobic. Due to this structural change, membrane protein is extracted, holes are opened in the cell membranes, and water enters the cells, so that the tumor cells can be ruptured and destroyed. IR700 is excited by receiving the near-infrared rays, and emits fluorescence having a wavelength different from an excitation wavelength. IR700 emits fluorescence having, for example, a wavelength of 704 nm when excited by receiving near-infrared rays having a wavelength of 689 nm. IR700 changes a structure of the IR700 while emitting the fluorescence by a photoreaction, and when IR700 destroys the tumor cells and the IR700 acts as a drug, IR700 stops emitting the fluorescence.

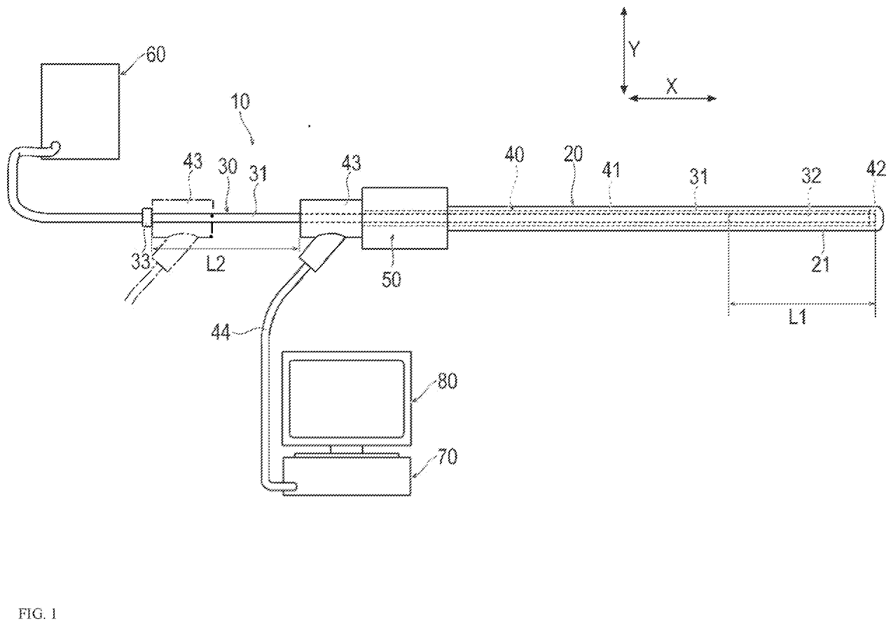

The treatment apparatus 10 illustrated in FIG. 1 can treat, with one device, a tumor in a cervix U (for example, cervical cancer) in a wide range A, which includes the cervix U, an external uterine ostium O, a uterine vagina UV around the external uterine ostium O, a vaginal vault VF, and a site closer to the vaginal vault VF on a vaginal introitus side than the vaginal vault VF of a vagina V, as illustrated in FIG. 5, and a tumor in the vagina V (for example, vaginal cancer). The treatment apparatus 10 can irradiate the antibody-photosensitive substance adsorbed on tumor cells C in a wide range, for example, from the cervix U to the vagina V with the excitation light.

A uterus is positioned behind the vagina V, an upper portion of the uterus is connected to left and right fallopian tubes, and the external uterine ostium O at a lower portion of the uterus is connected to the vagina V. The uterus is roughly divided into a uterine corpus and the cervix U, and the cervix U includes a cervical canal CC connected to the external uterine ostium O. The vagina V includes the vaginal vault VF that extends around the external uterine ostium O.

First, the treatment apparatus 10 according to the present embodiment will be described.

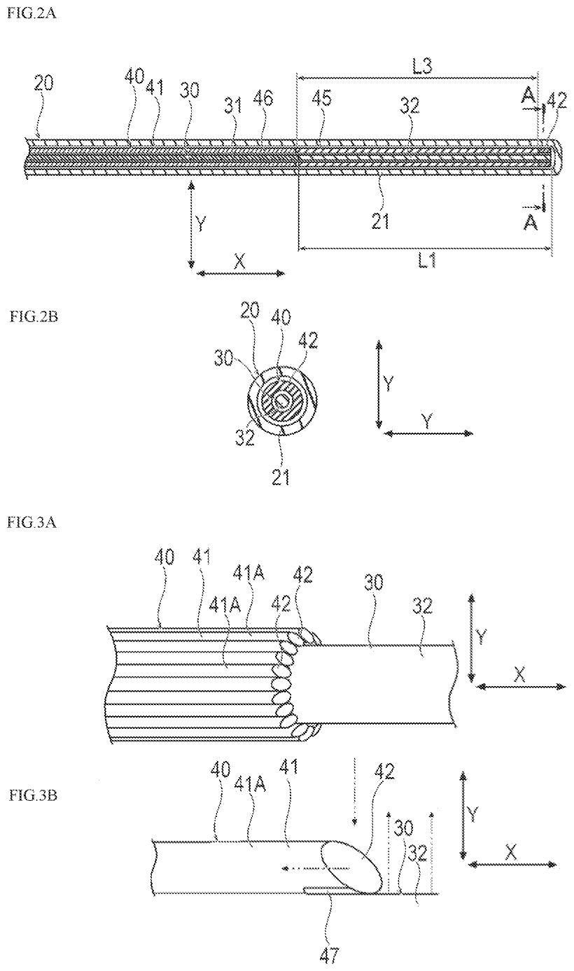

As illustrated in FIGS. 1 and 2, the treatment apparatus 10 includes an elongated tubular shaft 20 including a distal portion and a proximal portion, an elongated light irradiation unit 30 that emits the excitation light of the antibody-photosensitive substance, a side direction fluorescence detection unit 40 that detects fluorescence emitted by the excited antibody-photosensitive substance, an operation portion 50 interlocked with the proximal portion of the shaft 20, a light output device 60, a light-receiving device 70, and a display device 80.

The shaft 20 is a tubular shaped body extending from the operation portion 50 in a distal direction. The shaft 20 can accommodate the light irradiation unit 30 and the side direction fluorescence detection unit 40 in a lumen of the shaft 20. The proximal portion of the shaft 20 is fixed to the operation portion 50. The lumen of the shaft 20 is closed and sealed at a distal end of the shaft 20. The lumen of the shaft 20 is opened at a proximal end of the shaft 20 to accommodate the side direction fluorescence detection unit 40. The shaft 20 can be a circular tube extending linearly, but may be bent or may be not a circular tube.

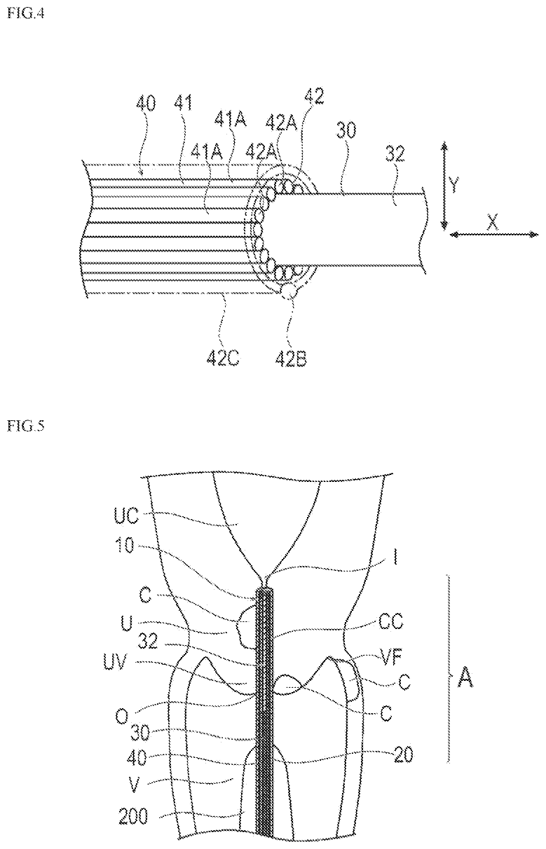

The shaft 20 includes a light transmitting portion 21 that is capable of transmitting the excitation light and the fluorescence at the distal portion of the shaft 20. The light transmitting portion 21 is disposed at least in a predetermined range of the distal portion of the shaft 20. At least a part of the light transmitting portion 21 is a site to be inserted into the cervical canal CC from the external uterine ostium O (see FIG. 4). The light transmitting portion 21 can be formed of a transparent or translucent material having optical transparency that allows the excitation light having a wavelength emitted by the light irradiation unit 30 and the fluorescence having a wavelength emitted by the antibody-photosensitive substance to be transmitted. A site of the shaft 20 on a proximal side relative to the light transmitting portion 21 may or may not be transparent. The entire shaft 20 may be implemented by the light transmitting portion 21.

The shaft 20 preferably has a certain degree of rigidity such that an operator can hold the operation portion 50 and push the shaft 20 to a desired position. A constituent material for the light transmitting portion 21 is not particularly limited, and the material for the light transmitting portion 21 can include, a resin represented by polymethyl methacrylate, polyethylene terephthalate, polycarbonate, polytetrafluoroethylene, or the like, glass, and the like. It is more preferable that a material for the shaft 20 has elasticity that allows the shaft 20 to be deformed while being bent along the cervical canal CC after being inserted into the cervical canal CC. Accordingly, it is possible to cope with individual differences in a shape of a cervical canal, and it is possible to reduce relative burden on an inner surface of the cervical canal and to further improve adhesion to the inner surface of the cervical canal.

A site of the shaft 20 on the proximal side relative to the light transmitting portion 21 may be formed of the same material as that of the light transmitting portion 21, or may be formed of a different material. In the present embodiment, the site of the shaft 20 on the proximal side relative to the light transmitting portion 21 may be formed of a material that cannot transmit the excitation light or the fluorescence, and the site of the shaft 20 on the proximal side can be formed of, for example, a metal represented by stainless steel, aluminum, titanium alloys, tin, magnesium alloys, or the like, a resin represented by polyetheretherketone (PEEK), polyamide, acrylonitrile butadiene styrene (ABS), polycarbonate, polyacetal, polyimide, or the like. An outer diameter of the shaft 20 is not particularly limited, and can be, for example, 0.5 mm to 6 mm. A length of the shaft 20 in an axial direction X is not particularly limited, and can be, for example, 100 mm to 400 mm. A length of the light transmitting portion 21 in the axial direction X is not particularly limited, and can be, for example, 10 mm to 50 mm.

The light irradiation unit 30 includes an elongated irradiation optical waveguide 31, a light emitting unit 32 disposed on a distal side of the irradiation optical waveguide 31, and a stopper 33 disposed on an outer peripheral surface of the irradiation optical waveguide 31. A part of the light irradiation unit 30 is movable in the lumen of the shaft 20 along the axial direction X of the shaft 20.

The irradiation optical waveguide 31 can be an elongated wire that propagates light. The irradiation optical waveguide 31 can be formed of, for example, one optical fiber. Note that the irradiation optical waveguide 31 may be formed of a plurality of optical fibers. A proximal portion of the irradiation optical waveguide 31 can be connected to the light output device 60 that outputs light. The irradiation optical waveguide 31 receives the near-infrared rays from the light output device 60 and can propagate the near-infrared rays to the light emitting unit 32. Note that the light irradiation unit 30 may be formed by an optical waveguide other than the optical fiber.

The stopper 33 is a site that protrudes radially outward from the outer peripheral surface of the irradiation optical waveguide 31 on the proximal side relative to the side direction fluorescence detection unit 40 that slides on the outer peripheral surface of the irradiation optical waveguide 31. The stopper 33 can be formed in a ring shape, for example, but a shape of the stopper 33 is not limited to a ring shape. The stopper 33 is disposed to restrict a movement of the side direction fluorescence detection unit 40, which slides on the outer peripheral surface of the irradiation optical waveguide 31, in the axial direction X with respect to the irradiation optical waveguide 31.

The light emitting unit 32 can be a cylindrical diffuser that is connected to a cut end of the optical fiber of the irradiation optical waveguide 31 and diffuses or scatters light received from the optical fiber. The light emitting unit 32 may be formed integrally with the optical fiber forming the irradiation optical waveguide 31 by processing a surface or an inside of the optical fiber. The light emitting unit 32 may be a cut end of the optical fiber. The light emitting unit 32 may be formed by a mirror and/or a lens disposed at the cut end of the optical fiber. The light emitting unit 32 is capable of emitting the light propagated from the irradiation optical waveguide 31 in a side direction Y perpendicular to the axial direction X of the shaft 20 (or the axial direction X of the irradiation optical waveguide 31). Note that the light emitting unit 32 is capable of emitting the light propagated from the irradiation optical waveguide 31 in the side direction Y perpendicular to the axial direction X of the shaft 20 (or the axial direction X of the irradiation optical waveguide 31). Note that the light irradiation unit 30 may be capable of emitting the excitation light in a direction other than the side direction Y as long as the light irradiation unit 30 is capable of emitting the excitation light in the side direction Y. The light emitting unit 32 may be an LED or the like that emits light by electric power. The light emitting unit 32 preferably has a certain length L1 in the axial direction X such that the excitation light can be simultaneously emitted to a certain wide range. The length L1 is not particularly limited, and can be, for example, 10 mm to 400 mm. Note that the length L1 of the light emitting unit 32 may be shorter or longer than the above range.

The side direction fluorescence detection unit 40 includes an elongated detection optical waveguide 41, a detection unit 42 disposed on a distal side of the detection optical waveguide 41, an optical connector 43 disposed on a proximal side of the detection optical waveguide 41, and a connection optical waveguide 44 disposed on a proximal side of the optical connector 43.

The detection optical waveguide 41 is an elongated tubular member that propagates light. The detection optical waveguide 41 surrounds an outer peripheral surface of the light irradiation unit 30 and is slidable (movable) along the light irradiation unit 30 in the axial direction X. A distal end of the detection optical waveguide 41 is interlocked with the detection unit 42, and a proximal end of the detection optical waveguide 41 is interlocked with the optical connector 43. In the present embodiment, the detection optical waveguide 41 includes a first detection optical waveguide 45 on the distal side and a second detection optical waveguide 46 on the proximal side. The second detection optical waveguide 46 on the proximal side is formed by a tubular core serving as an optical path, and a cladding that covers an inner peripheral surface and an outer peripheral surface of the core and reflects the light propagated through the core. Accordingly, the second detection optical waveguide 46 can efficiently propagate the light. The first detection optical waveguide 45 on the distal side can be entirely formed by a transparent tube body. Although an efficiency of propagating light in a proximal direction of the first detection optical waveguide 45 is not as high as that of the second detection optical waveguide 46, the first detection optical waveguide 45 can transmit light in the radial direction. A length L3 of the first detection optical waveguide 45 along the axial direction X is not particularly limited, and can be preferably substantially equal to the length L1 of the light emitting unit 32 along the axial direction X.

The detection unit 42 is a member capable of propagating the fluorescence received from the side direction Y to the detection optical waveguide 41. The detection unit 42 can be, for example, a ring-shaped scatterer that surrounds the light irradiation unit 30 in a peripheral direction. The detection unit 42 may be, for example, a mirror and/or a lens.

The detection unit 42 may be formed integrally with the detection optical waveguide 41 by processing a surface or an inside of the detection optical waveguide 41, or may be a cut end of the detection optical waveguide 41. The detection unit 42 may be formed by a mirror and/or a lens disposed at the cut end of the detection optical waveguide 41. The detection unit 42 may be capable of detecting the fluorescence from a direction other than the side direction Y as long as the detection unit 42 can detect the fluorescence from the side direction Y. The detection unit 42 may be a sensor that converts light into an electrical signal. In the present embodiment, the side direction fluorescence detection unit 40 includes a conductive wire capable of transmitting the electrical signal to a proximal side of the detection unit 42.

As a modification of the side direction fluorescence detection unit 40, as illustrated in FIGS. 3A and 3B, the detection optical waveguide 41 may be formed by a plurality of optical fibers 41A arranged in the peripheral direction in a manner of surrounding at least a part of the light irradiation unit 30. In this embodiment, each detection unit 42 is disposed at a distal end of the optical fiber 41A. Each detection unit 42 can be, for example, an incident surface of an end on a distal side of the optical fiber 41A cut obliquely in a manner of facing the side direction Y. A reflector 47 may be disposed on an outer surface of each of the optical fibers 41A facing the outer peripheral surface of the light irradiation unit 30 in order to prevent the excitation light emitted from the light irradiation unit 30 from entering the detection unit 42. The respective detection units 42 arranged side by side in the peripheral direction can independently detect the fluorescence. Therefore, by using the side direction fluorescence detection unit 40 including the plurality of optical fibers 41A, a position of a lesion extent in the peripheral direction in which the tumor cells C are present can be specified when viewed from the shaft 20.

As another modification of the side direction fluorescence detection unit 40, as illustrated in FIG. 4, the side direction fluorescence detection unit 40 may include a plurality of optical fibers 41A arranged in the peripheral direction in a manner of surrounding at least a part of the light irradiation unit 30, a spherical scatterer 42B that can be disposed near the detection units 42, which are the ends of the plurality of optical fibers 41A on the distal side, and a support tube 42C that supports the scatterer 42B at a distal end. The support tube 42C is disposed between an outer peripheral surface of the detection optical waveguide 41 formed in a tubular shape by the plurality of optical fibers 41A and an inner peripheral surface of the shaft 20, that is, disposed between the outer peripheral surface of the detection optical waveguide 41 and the inner peripheral surface of the shaft 20. The support tube 42C extends from the operation portion 50 in the proximal direction. The operator can move, by rotating, with fingers, a site of the support tube 42C which is extends to the outside of the proximal portion, the scatterer 42B in the peripheral direction at a position where the ends of the plurality of optical fibers 41A on the distal side are arranged. Accordingly, the scatterer 42B that receives the fluorescence can propagate the fluorescence to the end of the near optical fiber 41A on the distal side.

As illustrated in FIG. 1, the optical connector 43 connects the proximal end of the detection optical waveguide 41 and a distal end of the connection optical waveguide 44. The optical connector 43 can propagate the light received from the detection optical waveguide 41 to the connection optical waveguide 44 that extends in the side direction Y. Therefore, the optical connector 43 forms an optical waveguide.

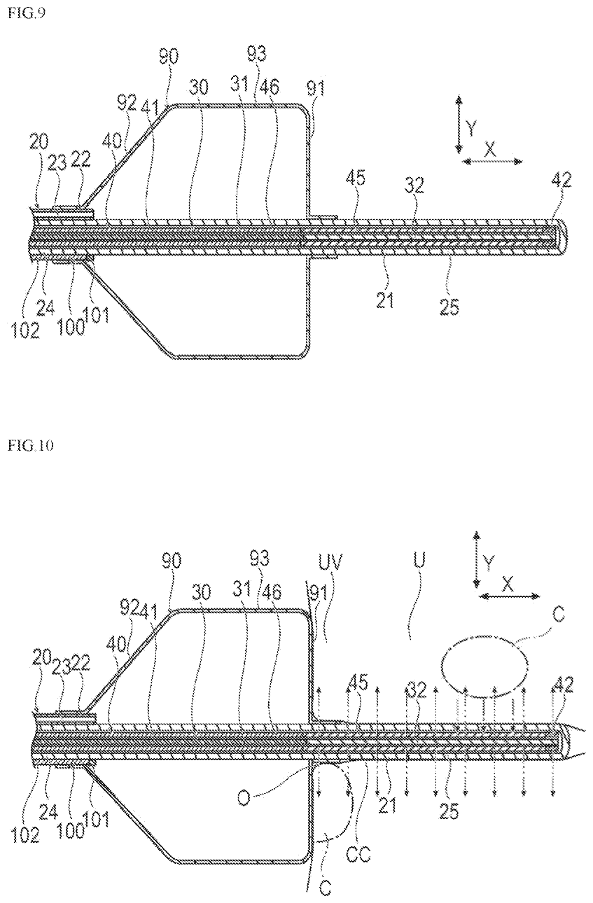

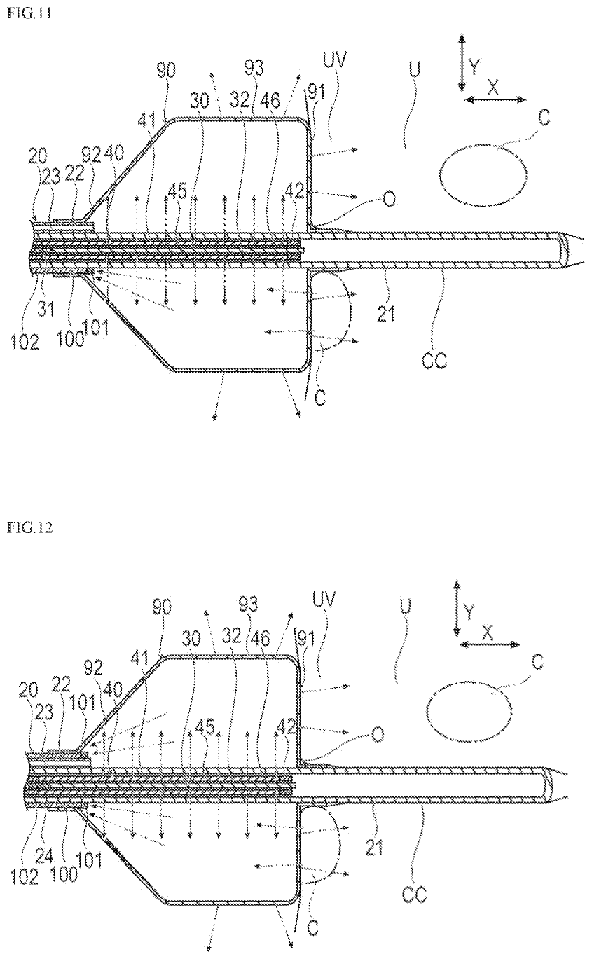

The connection optical waveguide 44 propagates the light propagated from the detection optical waveguide 41 to the light-receiving device 70 via the optical connector 43. When the detection optical waveguide 41 is a single optical waveguide, the connection optical waveguide 44 is also preferably a single optical waveguide. When the detection optical waveguide 41 is formed by the plurality of optical fibers 41A arranged in the peripheral direction, the connection optical waveguide 44 is also preferably formed by a plurality of optical waveguides (for example, a plurality of optical fibers). The optical connector 43 can independently connect the optical fibers 41A of the detection optical waveguide 41 to the respective optical fibers of the connection optical waveguide 44 that is formed by the plurality of optical fibers. As in a modification illustrated in FIG. 8, the detection optical waveguide 41 and the connection optical waveguide 44 may be integrally formed by the common optical fibers 41A. In this embodiment, in the detection optical waveguide 41, the optical fibers 41A, which are arranged in a manner of surrounding the outer peripheral surface of the light irradiation unit 30, extend as a bundle so as to be separated from the light irradiation unit 30 in the side direction Y, in such a manner that the light irradiation unit 30 passes through one of gaps of the bundle of optical fibers 41A, and form the connection optical waveguide 44. In this embodiment, the optical connector 43 is not provided.

As illustrated in FIG. 1, a proximal end of the optical connector 43 can come into contact with the stopper 33 disposed on the outer peripheral surface of the irradiation optical waveguide 31. A distance L2 along the axial direction X from a position of a proximal surface of the optical connector 43 when the detection unit 42 is disposed in a manner of surrounding a most distal end of the light emitting unit 32 to the stopper 33 is substantially consistent with the length L1 of the light emitting unit 32 along the axial direction X. Accordingly, the detection unit 42 is movable in the axial direction X within a range substantially equal to a range in which the excitation light from the light irradiation unit 30 can be emitted (i.e., a range from a distal end to a proximal end of the light emitting unit 32).

The operation portion 50 is a site to be held and operated by the operator. The proximal portion of the shaft 20 is fixed to the operation portion 50. The detection optical waveguide 41 extends in the proximal direction from a proximal portion of the operation portion 50. The irradiation optical waveguide 31 extends from the optical connector 43 at the proximal end of the detection optical waveguide 41. Note that a configuration of the operation portion 50 is not particularly limited.

The light output device 60 can output light having any wavelength to the irradiation optical waveguide 31 of the light irradiation unit 30 with any intensity (power) or energy. The light output device 60 outputs the excitation light, which is the near-infrared ray having a wavelength of, for example, 660 nm to 740 nm, to the irradiation optical waveguide 31 such that the light can be emitted at an intensity (power) of, for example, 1 mW to 5 W, and an energy of, for example, 1 Jcm⁻² to 50 Jcm⁻². The excitation light may be quantitatively emitted or intermittently pulse-emitted.

The light-receiving device 70 is connected with a proximal portion of the connection optical waveguide 44, and receives the fluorescence detected by the side direction fluorescence detection unit 40. The light-receiving device 70 can convert the received light into an electrical signal, perform predetermined arithmetic processing, and display a result on the display device 80 as image information. The light-receiving device 70 can include, for example, a computer including a storage circuit and an arithmetic circuit. The light-receiving device 70 may include a filter that removes the excitation light and leaves the fluorescence from the detected light. The filter includes a bandpass filter that leaves light having a wavelength of fluorescence, or a filter that specifies and removes pulse-emitted excitation light.

The display device 80 can be a monitor capable of displaying a visually recognizable image. The display device 80 is connected to the light-receiving device 70 such that a signal including image data can be received from the light-receiving device 70. The image displayed on the display device 80 can be a two-dimensional image, a three-dimensional image, or the like acquired by the side direction fluorescence detection unit 40. The display device 80 displays the image based on the image data received from the light-receiving device 70.

Next, a treatment method using the treatment apparatus 10 according to the first embodiment will be described.

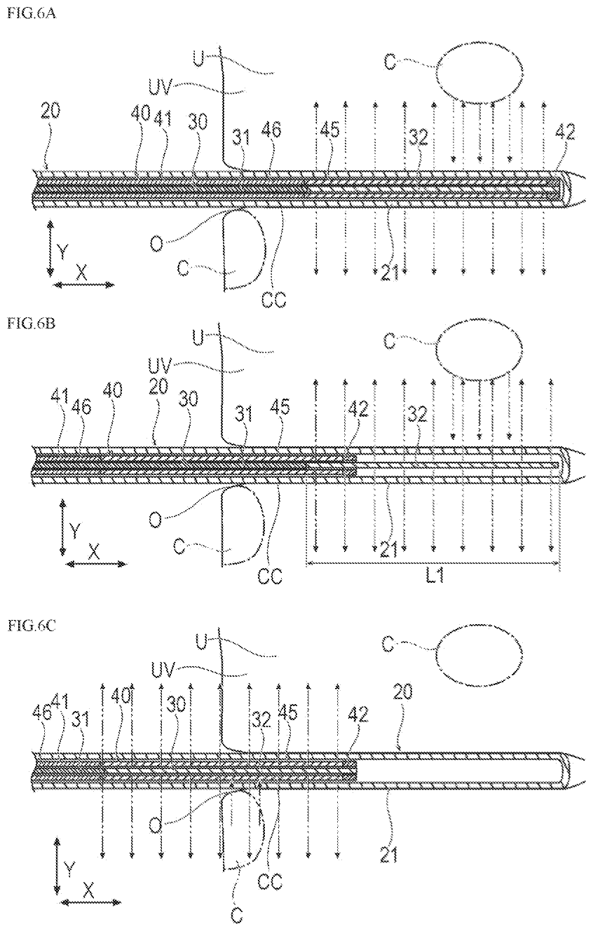

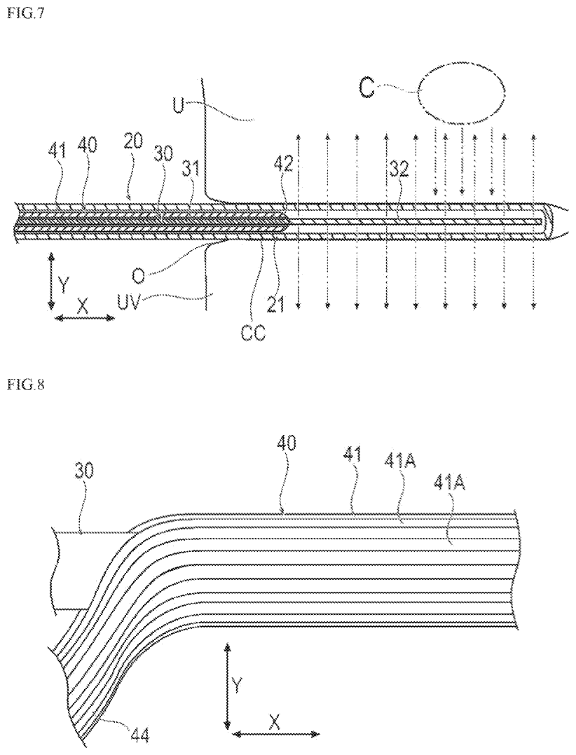

First, the antibody-photosensitive substance is administered into a body. A method for administering the antibody-photosensitive substance into the body is not particularly limited as long as the method can cause the antibody-photosensitive substance to reach the tumor cells C, and can be, for example, intravascular administration, and in the present embodiment, intravenous administration. After approximately 12 hours to 36 hours from the intravenous administration, as illustrated in FIGS. 5 and 6A, the operator opens a vaginal introitus by using a vaginal speculum 200, and inserts the shaft 20 of the treatment apparatus 10 into the vagina V from the vaginal introitus. The operator inserts the shaft 20 from the external uterine ostium O into the cervical canal CC while visually checking the distal portion of the shaft 20.

Next, the operator disposes the light emitting unit 32 of the light irradiation unit 30 inside the shaft 20. A position of the light emitting unit 32 can be, for example, a most distal position where the light emitting unit 32 is reachable in the lumen of the shaft 20, but is not limited to the most distal position. Thereafter, the operator operates the light output device 60 to supply the excitation light to the light irradiation unit 30. Accordingly, the light emitting unit 32 inside the shaft 20 can effectively emit the excitation light to the tumor cells C positioned in the cervix U. An irradiation direction of the excitation light from the light emitting unit 32 includes the side direction Y perpendicular to an axial center of the shaft 20. Therefore, the light emitting unit 32 can effectively emit the excitation light to the tumor cells C positioned in the cervix U from the cervical canal CC. The operator may cause the excitation light to be emitted while moving the light emitting unit 32 inside the shaft 20.

When the excitation light is emitted, the excitation light reaches the antibody-photosensitive substance adsorbed on the tumor cells C in the cervix U. Accordingly, a chemical change occurs in the antibody-photosensitive substance excited by the excitation light, and a structural change occurs in the antibody-photosensitive substance, which causes holes in the cell membranes. Accordingly, the tumor cells C irradiated with the excitation light are destroyed.

The operator detects, by the side direction fluorescence detection unit 40, the fluorescence emitted by the antibody-photosensitive substance excited by the excitation light while emitting the excitation light from the light emitting unit 32. Since the side direction fluorescence detection unit 40 can detect the fluorescence from the side direction Y, the fluorescence emitted by the tumor cells C positioned in the cervix U can be effectively detected from the cervical canal CC. As illustrated in FIG. 6B, the operator can detect the fluorescence while moving the detection unit 42 in the axial direction X within a range of the length L1 in which the light emitting unit 32 is disposed, while emitting the excitation light from the light emitting unit 32. The operator can specify (diagnose), based on the image displayed on the display device 80, a position of the tumor cells C in the axial direction X of the shaft 20 by specifying the fluorescence emitted by the excited antibody-photosensitive substance adsorbed on the tumor cells C. Therefore, the operator can intensively perform treatment or diagnosis of the lesion extent which is specified to have the tumor cells C. The light emitting unit 32 is covered by the detection unit 42 and the first detection optical waveguide 45. However, a length of the detection unit 42 along the axial direction X is relatively short with respect to the light emitting unit 32, and the first detection optical waveguide 45 can transmit light. Therefore, the excitation light emitted from the light emitting unit 32 can reach the antibody-photosensitive substance adsorbed on the tumor cells C with almost no influence from the detection unit 42 and the first detection optical waveguide 45. Therefore, the treatment apparatus 10 can simultaneously destroy the tumor cells C by emitting the excitation light and diagnose a lesion site by detecting the fluorescence. Note that the diagnosis includes the specification of the lesion extent in which the tumor cells C are present and check of destruction of the tumor cells C (check of disappearance or reduction).

When the detection unit 42 reaches the vicinity of the proximal end of the light emitting unit 32, the proximal surface of the optical connector 43 of the side direction fluorescence detection unit 40 abuts on the stopper 33 disposed on the outer peripheral surface of the irradiation optical waveguide 31. Accordingly, the detection unit 42 can be prevented from being separated from the light emitting unit 32.

Note that when the side direction fluorescence detection unit 40 is implemented by an aspect of the modification illustrated in FIG. 3, since the detection optical waveguide 41 is formed by the plurality of optical fibers 41A, the first detection optical waveguide 45 that transmits light in the radial direction cannot be provided in the detection optical waveguide 41. Therefore, when the detection unit 42 is disposed on the distal side relative to the proximal end of the light emitting unit 32, the excitation light emitted from a range of the light emitting unit 32 that is covered by the detection unit 42 or the detection optical waveguide 41 is hardly transmitted through the detection unit 42 or the detection optical waveguide 41, and cannot reach the tumor cells C. Therefore, when the excitation light is emitted to the antibody-photosensitive substance adsorbed on the tumor cells C by the light emitting unit 32, as illustrated in FIG. 7, the detection unit 42 is preferably retracted in the proximal direction such that the detection unit 42 and the detection optical waveguide 41 do not interfere with the emission from the light emitting unit 32. The detection of the fluorescence by the detection unit 42 is performed by moving the detection unit 42 to a predetermined position in the axial direction X only when the detection of the fluorescence is desired. Therefore, in the case of this modification, it may be difficult to continuously detect the fluorescence by the detection unit 42 during the emission of the excitation light. However, since the plurality of detection units 42 are arranged side by side in the peripheral direction, the position of the tumor cells C in the peripheral direction can be specified by independently using the plurality of detection units 42. Therefore, for example, even when different tumor cells C are present at a plurality of positions in the peripheral direction at a predetermined position in the axial direction X, the tumor cells C can be individually specified and appropriately treated.

Further, when the side direction fluorescence detection unit 40 is implemented by an aspect of the modification illustrated in FIG. 4, when the fluorescence enters the spherical scatterer 42B moving in the peripheral direction, the fluorescence is propagated from the scatterer 42B to the optical fiber 41A including an end 42A facing the scatterer 42B. Therefore, the position of the tumor cells C in the peripheral direction can be specified by using the scatterer 42B and the plurality of optical fibers 41A.

When the operator determines that the fluorescence disappears by the display device 80 or a predetermined time passes, the operator determines that the tumor cells C are sufficiently destroyed and stops emitting the excitation light. The case in which it is determined that the fluorescence disappears includes a case in which it is checked by the display device 80 that the fluorescence disappears or a case in which the fluorescence decreases to a preset threshold value or less (or less than the threshold value). Whether the tumor cells C are sufficiently destroyed may be determined by the operator, or may be automatically determined by a program or the like provided in the light-receiving device 70 or the like. The light-receiving device 70 may cause the display device 80 to display a result of the determination.

Next, as illustrated in FIG. 6C, the operator pulls the light irradiation unit 30 and moves the light emitting unit 32 in the proximal direction in a state in which a position of the shaft 20 is maintained. A distance for moving the light emitting portion 32 in the proximal direction is preferably equal to or less than the length L1 of the light emitting unit 32 along the axial direction X. Accordingly, occurrence of a range in which the excitation light cannot be emitted can be prevented.

Next, the operator operates the light output device 60 to supply the excitation light to the light irradiation unit 30. It is possible to emit the excitation light from the light emitting unit 32, detect the fluorescence by the detection unit 42, move the detection unit 42, specify the range of the tumor cells C, check the destruction of the tumor cells C, and stop emitting the excitation light, in the same procedure as before moving the light emitting unit 32 in the proximal direction. The operator can repeat once or more a series of procedures (i.e., repeated procedures) for moving the light emitting unit 32, emitting the excitation light from emitting unit 32, detecting the fluorescence by the detection unit 42, moving the detection unit 42, specifying the range of the tumor cells C, checking the destruction of the tumor cells C, and stopping emitting the excitation light. Note that a part of the method included in the repeated series of procedures may not be performed. For example, the emission of the excitation light may not be stopped during the repeated series of procedures.

As described above, the treatment apparatus 10 according to the first embodiment is the treatment apparatus 10 that detects and destroys the tumor cell C by irradiating the antibody-photosensitive substance adsorbed on a tumor cell membrane with the excitation light. The treatment apparatus includes: the elongated tubular shaft 20 having optical transparency; the light irradiation unit 30 configured to emit the excitation light of the antibody-photosensitive substance from an inside of the shaft 20 to the side direction Y perpendicular to the axial direction X of the shaft 20; and the side direction fluorescence detection unit 40 configured to detect, from the side direction Y perpendicular to the axial direction X of the shaft 20, fluorescence emitted by the excited antibody-photosensitive substance, and movable in the axial direction X with respect to the shaft 20.

According to the treatment apparatus 10 described above, since the side direction fluorescence detection unit 40 is movable in the axial direction X, the fluorescence emitted by the antibody-photosensitive substance excited by the excitation light from the light irradiation unit 30 can be detected by the side direction fluorescence detection unit 40 at a plurality of positions in the axial direction X. Therefore, according to the treatment apparatus 10, it is possible to minimally invasively specify the lesion extent in the axial direction X and check the progress of the treatment without excising a tissue while performing the treatment for destroying the tumor cells C. Note that the light irradiation unit 30 may be capable of emitting the excitation light in a direction other than the side direction Y as long as the light irradiation unit 30 is capable of emitting the excitation light in the side direction Y. The side direction fluorescence detection unit 40 may be capable of detecting the fluorescence from a direction other than the side direction Y as long as the side direction fluorescence detection unit 40 can detect the fluorescence from the side direction Y.

The side direction fluorescence detection unit 40 includes the ring-shaped scatterer disposed along the peripheral direction of the light irradiation unit 30 and movable in the axial direction X along the outer peripheral surface of the light irradiation unit 30, and the optical waveguide disposed on a proximal side of the scatterer. Accordingly, since the side direction fluorescence detection unit 40 can detect the fluorescence at the plurality of positions in the axial direction X, the lesion extent in the axial direction X in which the tumor cells C are present can be specified (i.e., diagnosed).

The side direction fluorescence detection unit 40 may include the plurality of optical fibers 41A, and a plurality of incident portions (for example, ends or lenses) of the optical fibers 41A may be disposed along the peripheral direction of the light irradiation unit 30, and may be movable in the axial direction X along the outer peripheral surface of the light irradiation unit 30. Accordingly, since the side direction fluorescence detection unit 40 can individually detect the fluorescence at a plurality of positions in the peripheral direction, the lesion extent in the peripheral direction in which the tumor cells C are present can be specified (i.e., diagnosed).

Note that the treatment apparatus 10 according to the first embodiment may be used for treatment of an organ other than the cervix U. For example, the treatment apparatus 10 can be used for treatment of an elongated tubular organ such as a ureter, a prostate, or a blood vessel.

The treatment method according to the first embodiment is a treatment method for detecting and destroying the tumor cell C by irradiating the antibody-photosensitive substance adsorbed on the tumor cell membrane of the cervix U with the excitation light. The treatment method includes: administering the antibody-photosensitive substance into the body; after the administration, widening the vagina V by the vaginal speculum 200, and inserting the treatment apparatus 10 into the vagina V, the treatment apparatus 10 including the elongated tubular shaft 20 having optical transparency, the light irradiation unit 30 configured to emit the excitation light of the antibody-photosensitive substance, and the side direction fluorescence detection unit 40 configured to detect, from the side direction Y perpendicular to the axial direction X of the shaft 20, the fluorescence emitted by the excited antibody-photosensitive substance; inserting the shaft 20 into the cervical canal CC while visually checking the distal portion of the shaft 20 and the external uterine ostium O; emitting the excitation light by the light irradiation unit 30 from the inside of the shaft 20 in the side direction Y; and while emitting the excitation light, detecting the fluorescence emitted by the antibody-photosensitive substance by moving the side direction fluorescence detection unit 40 in the axial direction X with respect to the shaft 20, and checking disappearance or reduction of the fluorescence.

According to the treatment method described above, since the fluorescence is detected by moving the side direction fluorescence detection unit 40 in the axial direction X while emitting the excitation light from the inside of the cervical canal, it is possible to specify or diagnose the position of the lesion extent along the axial direction X and check the progress of the treatment without excising a tissue while performing the treatment for destroying the tumor cells C of the cervix U.

In this treatment method, the repeated procedures may be repeated once or more after checking the disappearance or reduction of the fluorescence, the repeated procedures including moving the light irradiation unit 30 in the axial direction X, and while emitting the excitation light by the light irradiation unit 30 from the inside of the shaft 20 in the side direction Y, detecting the fluorescence emitted by the antibody-photosensitive substance by moving the side direction fluorescence detection unit 40 in the axial direction X with respect to the shaft 20, and checking the disappearance or reduction of the fluorescence. Accordingly, this treatment method makes it possible to minimally invasively treat the tumor in a wide range along the axial direction X of the shaft 20, specify the lesion extent, and check the progress of the treatment without excising a tissue.

### Second Embodiment

As illustrated in FIG. 9, the treatment apparatus 10 according to a second embodiment is different from that of the first embodiment in that the treatment apparatus 10 according to the second embodiment is provided with a light adjustment unit 90 configured to change a direction of excitation light emitted from the light irradiation unit 30 and a distal direction fluorescence detection unit 100 configured to detect fluorescence from a distal direction of the shaft 20.

The shaft 20 includes an inflation tube 22 on which an inflation lumen 23 is formed. The inflation lumen 23 communicates with an inside of the light adjustment unit 90. A proximal portion of the inflation tube 22 is positioned at the operation portion 50, and is connectable to a syringe or an indeflator that supplies an inflation fluid.

The light adjustment unit 90 is a member that is disposed on an outer periphery of a distal portion of the shaft 20, inflated radially outward in the vagina V, and allows light to be emitted in a wide range of the vagina V. The light adjustment unit 90 can transmit the excitation light to an outside while changing the direction of the excitation light emitted from the light emitting unit 32 disposed in a lumen of the shaft 20 passing through the inside of the light adjustment unit 90. Therefore, the light adjustment unit 90 can be formed of a transparent or translucent material capable of transmitting light having wavelengths of the excitation light and the fluorescence.

A distal portion of the light adjustment unit 90 is fixed to an outer peripheral surface of the shaft 20 closer to a proximal side than a most distal end of the shaft 20, and a proximal portion of the light adjustment unit 90 is fixed to the outer peripheral surface of the shaft 20 further closer to the proximal side. The inside of the light adjustment unit 90 communicates with the inflation lumen 23 provided on the shaft 20. The light adjustment unit 90 is a balloon that can be deformed and inflated when a fluid flows into the inside of the light adjustment unit 90.

The light adjustment unit 90 includes a distal end light adjustment unit 91 on the distal side, a proximal end light adjustment unit 92 on the proximal side, and an intermediate light adjustment unit 93 disposed between the proximal end light adjustment unit 92 and the distal end light adjustment unit 91. The distal end light adjustment unit 91 is fixed to the outer peripheral surface of the shaft 20. In an inflated state of the light adjustment unit 90, the distal end light adjustment unit 91 forms a plane facing the distal side. The distal end light adjustment unit 91 is substantially perpendicular to the axial center of the shaft 20, and abuts against the uterine vagina UV around the external uterine ostium O (see FIG. 10). Note that a shape of the distal end light adjustment unit 91 may be not a planar shape. The intermediate light adjustment unit 93 has a cylindrical shape having a substantially constant outer diameter in an axial center direction between the proximal end light adjustment unit 92 and the distal end light adjustment unit 91. Note that the intermediate light adjustment unit 93 may not be formed with a substantially constant outer diameter.

A proximal portion of the proximal end light adjustment unit 92 is fixed to the outer peripheral surface of the shaft 20 on the proximal side relative to the distal end light adjustment unit 91. In the inflated state, an outer diameter of the proximal end light adjustment unit 92 increases in a tapered shape toward the distal side. A distal portion of the proximal end light adjustment unit 92 is interlocked with a proximal portion of the intermediate light adjustment unit 93. The tapered proximal end light adjustment unit 92 helps prevent the light adjustment unit 90 from being pushed in the proximal side and deformed when the distal end light adjustment unit 91 abuts against the uterine vagina UV and receives a reaction force in a proximal direction (see FIG. 10). Note that the proximal end light adjustment unit 92 may not be formed in a tapered shape.

A constituent material for the light adjustment unit 90 is not particularly limited as long as the constituent material has a certain degree of flexibility and can transmit the excitation light emitted from the light irradiation unit 30 and the fluorescence emitted by an antibody-photosensitive substance, and the material for the light adjustment unit 90 can be, for example, silicone, polyamide, polyethylene terephthalate, urethane, and the like. A maximum outer diameter of the light adjustment unit 90 when being inflated is not particularly limited, and can be, for example, 5 mm to 50 mm. A length of the light adjustment unit 90 in the axial direction X when being inflated is not particularly limited, and can be, for example, 10 mm to 60 mm. The light adjustment unit 90 is capable of scattering, diffusing, or reflecting the excitation light emitted in the side direction Y by the light emitting unit 32 disposed inside to adjust the excitation light in a direction different from the side direction Y (in particular, the distal direction). Therefore, it is possible to emit the light to a wide range through the light adjustment unit 90 even in a range other than a range directly irradiated with the light from the light emitting unit 32.

The light adjustment unit 90 may be formed in various shapes. It is preferable that the light adjustment unit 90 is appropriately selectable according to shapes of the uterine vagina UV, the vaginal vault VF, or the vagina V of a patient.

The shaft 20 includes a base shaft 24 having a distal portion at which the light adjustment unit 90 is disposed, and a distal shaft 25 protruding from the light adjustment unit 90 in the distal direction. The light transmitting portion 21 having optical transparency is positioned at the distal shaft 25 and at least a part of a site of the base shaft 24 that is disposed inside the light adjustment unit 90.

The distal direction fluorescence detection unit 100 includes a second detection unit 101 that is disposed inside the light adjustment unit 90 and capable of detecting a two-dimensional image, and a cable 102 that transmits information on the two-dimensional image detected by the second detection unit 101 to the light-receiving device 70. The second detection unit 101 is disposed inside the light adjustment unit 90 and outside the shaft 20. The cable 102 is introduced from the outside to the inside of the light adjustment unit 90 through between the outer peripheral surface of the shaft 20 and an inner peripheral surface of the proximal end light adjustment unit 92. The second detection unit 101 can be, for example, a small CCD (charge-coupled device) image sensor or a CMOS (complementary metal-oxide semiconductor) image sensor. In this embodiment, the cable 102 is a conductive wire that transmits an electrical signal. The second detection unit 101 may be ends of a plurality of optical fibers forming a two-dimensional array. In this embodiment, the cable 102 is formed by the plurality of optical fibers. A wide-angle lens or the like may be disposed in the sensor or the optical fiber.

As in a modification illustrated in FIG. 12, the distal direction fluorescence detection unit 100 may include two or more second detection units 101 arranged at different positions in a peripheral direction. Accordingly, the distal direction fluorescence detection unit 100 can detect a wide range with two-dimensional images to specify a position of a lesion in the wide range and check progress of treatment. Note that although the second detection unit 101 and the inflation tube 22 overlap each other in FIG. 12, the second detection unit 101 and the inflation tube 22 may be disposed at different positions in the peripheral direction without overlapping.

Next, a treatment method using the treatment apparatus 10 according to the second embodiment will be described.

First, the antibody-photosensitive substance is administered into a body by intravenous administration, for example. After approximately 12 hours to 36 hours from the intravenous administration, an operator opens a vaginal introitus by using the vaginal speculum 200, and inserts the distal shaft 25 of the treatment apparatus 10 into the vagina V from the vaginal introitus. The operator inserts the distal shaft 25 from the external uterine ostium O into the cervical canal CC while visually checking the distal portion of the shaft 20 and the external uterine ostium O. At this time, since the light adjustment unit 90 is not inflated, the operator can rather easily insert the distal shaft 25 into the cervical canal CC.

Next, the operator connects the syringe or the indeflator that accommodates the fluid such as air or a saline solution to the proximal portion of the inflation tube 22, and supplies the inflation fluid into the light adjustment unit 90 via the inflation lumen 23. Accordingly, the light adjustment unit 90 is inflated in the vagina V. Note that the light adjustment unit 90 is preferably inflated to such a degree that the light adjustment unit 90 is movable in the vagina V. Next, the operator pushes the operation portion 50, and presses a surface of the inflated light adjustment unit 90 on the distal side against the uterine vagina UV. The uterine vagina UV is a site on a vagina V side of the cervix U, and on which the external uterine ostium O is formed. When the light adjustment unit 90 comes into close contact with the uterine vagina UV, the distal shaft 25 is positioned within the cervical canal CC, and the light adjustment unit 90 is positioned within the vagina V. Next, the operator further inflates the light adjustment unit 90. Accordingly, the light adjustment unit 90 is inflated in a state of being in relatively close contact with the uterine vagina UV. The light adjustment unit 90 is inflated in accordance with a shape of an organ.

Next, the operator disposes the light emitting unit 32 of the light irradiation unit 30 inside the distal shaft 25. A position of the light emitting unit 32 can be, for example, a most distal position where the light emitting unit 32 is reachable in a lumen of the distal shaft 25, but is not limited to the most distal position. Thereafter, the operator operates the light output device 60 to supply the excitation light to the light irradiation unit 30. Accordingly, the light emitting unit 32 inside the shaft 20 can effectively emit the excitation light to the tumor cells C positioned in the cervix U. Similarly to the method described in the first embodiment, the operator can emit the excitation light from the inside of the shaft 20 inserted into the cervical canal CC, detect the fluorescence by the detection unit 42, emit the excitation light from the light emitting unit 32, detect the fluorescence by the detection unit 42, move the detection unit 42, specify a lesion extent in which the tumor cells C are present, check destruction of the tumor cells C, and stop emitting the excitation light. Similarly to the method described in the first embodiment, the operator can perform the above series of procedures (i.e., repeated procedures) once or more inside the cervical canal CC.

Thereafter, the operator pulls the light irradiation unit 30 and the side direction fluorescence detection unit 40 in a state in which the position of the shaft 20 is maintained, and moves the light emitting unit 32 and the detection unit 42 to the inside of the light adjustment unit 90. Next, the operator operates the light output device 60 to supply the excitation light to the light irradiation unit 30. Accordingly, the entire light adjustment unit 90 that receives the light from the light emitting unit 32 emits light. That is, a part of the excitation light that reaches the light adjustment unit 90 is transmitted through the light adjustment unit 90, is scattered, diffused, or reflected by the light adjustment unit 90, and then emitted to a wide range including the distal direction. Therefore, the light emitting unit 32 and the light adjustment unit 90 can effectively emit the excitation light to the tumor cells C positioned mainly at the external uterine ostium O, the uterine vagina UV, the vaginal vault VF, and a site that is near the vaginal vault VF and is on a vaginal introitus side relative to the vaginal vault VF of the vagina V. Note that the operator may emit the excitation light while moving the light emitting unit 32 inside the light adjustment unit 90. When the light emitting unit 32 is elongated in the axial direction X and can emit light simultaneously from both the distal shaft 25 and the light adjustment unit 90, the operator does not need to move the light emitting unit 32 between the distal shaft 25 and the light adjustment unit 90.

When the excitation light is emitted, the excitation light mainly reaches the antibody-photosensitive substance adsorbed on the tumor cells C in the external uterine ostium O, the uterine vagina UV, the vaginal vault VF, and the site that is near the vaginal vault VF and is on the vaginal introitus side relative to the vaginal vault VF of the vagina V. Accordingly, a chemical change occurs in the antibody-photosensitive substance that receives the excitation light, and a structural change occurs in the antibody-photosensitive substance, which causes holes in cell membranes. Accordingly, the tumor cells C irradiated with the excitation light are destroyed.

The operator detects, by the distal direction fluorescence detection unit 100, the fluorescence emitted by the antibody-photosensitive substance excited by the excitation light while emitting the excitation light from the light emitting unit 32. Note that the light emitting unit 32 may or may not be covered by the detection unit 42 or the first detection optical waveguide 45 as long as the light emitting unit 32 can emit light to the inside of the light adjustment unit 90. Since the second detection unit 101 of the distal direction fluorescence detection unit 100 can detect the fluorescence from the distal direction, it is possible to effectively detect the fluorescence emitted by the antibody-photosensitive substance adsorbed on the tumor cells C in the external uterine ostium O, the uterine vagina UV, the vaginal vault VF, and the site that is near the vaginal vault VF and is on the vaginal introitus side relative to the vaginal vault VF of the vagina V. The operator can specify, based on the two-dimensional image displayed on the display device 80, a position of the tumor cells C positioned on a distal direction side of the light adjustment unit 90 by specifying the fluorescence emitted by the excited antibody-photosensitive substance adsorbed on the tumor cells C. The treatment apparatus 10 can simultaneously destroy the tumor cells C by emitting the excitation light from the light emitting unit 32 and diagnose a lesion site by detecting the fluorescence by the second detection unit 101.

When the operator determines that the fluorescence disappears by the display device 80 or a predetermined time passes, the operator determines that the tumor cells C are sufficiently destroyed and stops emitting the excitation light. Thereafter, the operator deflates the light adjustment unit 90, and draws the treatment apparatus 10 out of the cervical canal CC and the vagina V. Accordingly, this treatment method ends.

As described above, the treatment apparatus 10 according to the second embodiment includes: the light adjustment unit 90 configured to change the direction of the excitation light emitted from the light irradiation unit 30; and the distal direction fluorescence detection unit 100 configured to detect the fluorescence from the distal direction of the shaft 20, in which the shaft 20 includes the base shaft 24 having the distal portion at which the light adjustment unit 90 is disposed, and the distal shaft 25 protruding from the light adjustment unit 90 in the distal direction and having optical transparency, and the light irradiation unit 30 is movable between the inside of the distal shaft 25 and the inside of the light adjustment unit 90. Accordingly, by disposing the light irradiation unit 30 inside the distal shaft 25, it is possible to detect the fluorescence from the side direction Y by the side direction fluorescence detection unit 40 while emitting the excitation light in the side direction Y. Further, by disposing the light irradiation unit 30 inside the light adjustment unit 90, it is possible to detect the fluorescence from the distal direction by the distal direction fluorescence detection unit 100 while emitting the excitation light in the distal direction. Therefore, a tumor in the cervix U can be effectively treated by emitting the excitation light from the inside of the cervical canal CC by disposing the light irradiation unit 30 inside the distal shaft 25 inserted into the cervical canal CC, and the tumor in the cervix U can be effectively treated by emitting the excitation light from the vagina V side by disposing the light irradiation unit 30 inside the light adjustment unit 90 that is disposed at the site close to the uterine vagina UV in the vagina V. The light adjustment unit 90 can be, for example, a balloon, but may not be a balloon. The light adjustment unit 90 may be, for example, a member that is not inflated and deflated in a radial direction.

The treatment method according to the second embodiment is a treatment method for detecting and destroying the tumor cell C by irradiating the antibody-photosensitive substance adsorbed on the cell membrane of the tumor developed in at least a part in a range from the cervix U to the vagina V with the excitation light. The treatment method includes: administering the antibody-photosensitive substance into the body; after the administration, widening the vagina V by the vaginal speculum 200 and inserting the treatment apparatus 10 into the vagina V, the treatment apparatus 10 including the elongated base shaft 24, the light adjustment unit 90 disposed at the distal portion of the base shaft 24 and configured to change the direction of light, the elongated tubular distal shaft 25 protruding from the light adjustment unit 90 in the distal direction and having optical transparency, the light irradiation unit 30 configured to emit the excitation light of the antibody-photosensitive substance, the side direction fluorescence detection unit 40 configured to detect, from the side direction Y perpendicular to the axial direction X of the distal shaft 25, the fluorescence emitted by the excited antibody-photosensitive substance, and the distal direction fluorescence detection unit 100 configured to detect the fluorescence from the distal side; inserting the distal shaft 25 into the cervical canal CC while visually checking the distal shaft 25 and the external uterine ostium O; inflating the light adjustment unit 90 in accordance with the shape of the organ; emitting the excitation light by the light irradiation unit 30 in the side direction Y from the inside of the distal shaft 25; while emitting the excitation light, detecting the fluorescence emitted by the antibody-photosensitive substance by moving the side direction fluorescence detection unit 40 in the axial direction X with respect to the distal shaft 25, and checking disappearance or reduction of the fluorescence; moving the light irradiation unit 30 to a position where the light adjustment unit 90 is disposed in the axial direction X; and while emitting the excitation light through the light adjustment unit 90, detecting the fluorescence emitted by the antibody-photosensitive substance by the distal direction fluorescence detection unit 100, and checking the disappearance or reduction of the fluorescence.

Accordingly, in this treatment method, by emitting the excitation light from the inside of the cervical canal CC and detecting the fluorescence by the side direction fluorescence detection unit 40, it is possible to effectively treat the tumor in the cervix U, specify the lesion extent, and check the progress of the treatment without excising a tissue. Further, by emitting the excitation light from the inside of the light adjustment unit 90 disposed at the site close to the uterine vagina UV in the vagina V and detecting the fluorescence by the distal direction fluorescence detection unit 100, it is possible to minimally invasively treat the tumor distributed in the uterine vagina UV, which is the cervix U around the external uterine ostium O close to the vagina V, specify the lesion extent, and check the progress of the treatment without excising a tissue. Note that the light irradiation unit 30 may be capable of emitting the excitation light in a direction other than the side direction Y as long as the light irradiation unit 30 is capable of emitting the excitation light in the side direction Y. The side direction fluorescence detection unit 40 may be capable of detecting the fluorescence from a direction other than the side direction Y as long as the side direction fluorescence detection unit 40 can detect the fluorescence from the side direction Y.

### Third Embodiment

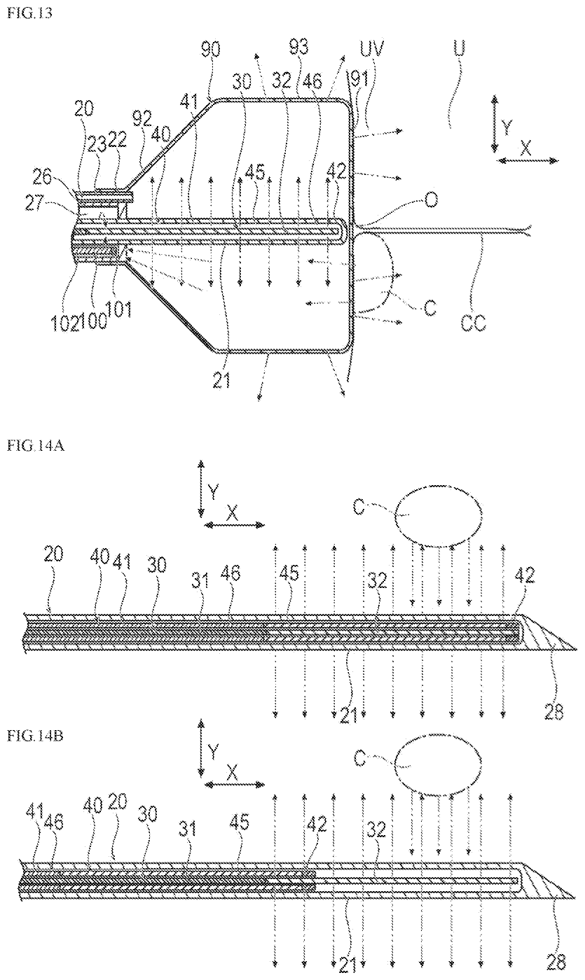

As illustrated in FIG. 13, the treatment apparatus 10 according to a third embodiment is different from that of the second embodiment in that the distal shaft 25 is not provided on the shaft 20, the detection unit 42 that is movable in the axial direction X is not provided, and the second detection unit 101 is movable in a peripheral direction.

The shaft 20 includes the light transmitting portion 21 capable of transmitting excitation light and fluorescence inside the light adjustment unit 90. The distal end light adjustment unit 91 is closed without the shaft 20 passing through. Note that a distal side of the light adjustment unit 90 may be a structure that allows the light adjustment unit 90 to be fixed with respect to a central axis. Accordingly, a shape and a position of the light adjustment unit 90, which is a balloon, can be rather easily maintained. The shaft 20 includes a tubular detection shaft 26 that is interlocked with the proximal end light adjustment unit 92 and includes a detection lumen 27 that does not communicate with a lumen of the light adjustment unit 90. The second detection unit 101 is disposed inside the detection lumen 27.

The distal direction fluorescence detection unit 100 is disposed inside the detection lumen 27 in a manner of being rotatable about an axis of the shaft 20. The second detection unit 101 disposed at a distal portion of the distal direction fluorescence detection unit 100 can detect the fluorescence from the distal direction as a two-dimensional image. The second detection unit 101 may be disposed, for example, at a distal portion of a tubular member in a manner of being rotatable inside the detection lumen 27.

Next, a treatment method using the treatment apparatus 10 according to the third embodiment will be described.

First, an antibody-photosensitive substance is administered into a body, for example, by intravenous administration. After approximately 12 hours to 36 hours from the intravenous administration, an operator opens a vaginal introitus by using the vaginal speculum 200, and inserts a distal end of the treatment apparatus 10 into the vagina V from the vaginal introitus.

Next, the operator connects a syringe or an indeflator that accommodates a fluid such as air or a saline solution to a proximal portion of the inflation tube 22, and supplies an inflation fluid into the light adjustment unit 90 via the inflation lumen 23. Accordingly, the light adjustment unit 90 is inflated in the vagina V. Note that the light adjustment unit 90 is preferably inflated to such a degree that the light adjustment unit 90 is movable in the vagina V. Next, the operator pushes the operation portion 50, and presses a surface of the inflated light adjustment unit 90 on the distal side against the uterine vagina UV. Next, the operator further inflates the light adjustment unit 90. Accordingly, the light adjustment unit 90 is inflated in a state of being in relatively close contact with the uterine vagina UV. The light adjustment unit 90 is inflated according to a shape of an organ.

Next, the operator disposes the light emitting unit 32 of the light irradiation unit 30 inside the light adjustment unit 90 of the shaft 20. Thereafter, the operator operates the light output device 60 to supply the excitation light to the light irradiation unit 30. Accordingly, the entire light adjustment unit 90 that receives the light from the light emitting unit 32 emits light. That is, a part of the excitation light that reaches the light adjustment unit 90 passes through the light adjustment unit 90, and is scattered, diffused, or reflected by the light adjustment unit 90, and then emitted to a wide range including the distal direction. Therefore, the light emitting unit 32 and the light adjustment unit 90 can effectively emit the excitation light to the tumor cells C positioned mainly at the external uterine ostium O, the uterine vagina UV, the vaginal vault VF, and a site that is near the vaginal vault VF and is on a vaginal introitus side relative to the vaginal vault VF of the vagina V. Note that the operator may emit the excitation light while moving the light emitting unit 32 inside the light adjustment unit 90.

When the excitation light is emitted, the excitation light mainly reaches the antibody-photosensitive substance adsorbed on the tumor cells C in the external uterine ostium O, the uterine vagina UV, the vaginal vault VF, and the site that is near the vaginal vault VF and is on the vaginal introitus side relative to the vaginal vault VF of the vagina V. Accordingly, a chemical change occurs in the antibody-photosensitive substance that receives the excitation light, and a structural change occurs in the antibody-photosensitive substance, which causes holes in cell membranes. Accordingly, the tumor cells C irradiated with the excitation light are destroyed.

The operator detects, by the distal direction fluorescence detection unit 100, the fluorescence emitted by the antibody-photosensitive substance excited by the excitation light while emitting the excitation light from the light emitting unit 32. The operator can detect the fluorescence while moving the second detection unit 101 in the peripheral direction around the axis of the shaft 20. Since the second detection unit 101 of the distal direction fluorescence detection unit 100 can detect the fluorescence from the distal direction, it is possible to effectively detect the fluorescence emitted by the antibody-photosensitive substance adsorbed on the tumor cells C in the external uterine ostium O, the uterine vagina UV, the vaginal vault VF, and the site that is near the vaginal vault VF and is on the vaginal introitus side relative to the vaginal vault VF of the vagina V. The operator can specify, based on the two-dimensional image displayed on the display device 80, a position of the tumor cells C positioned on the distal direction side of the light adjustment unit 90 by specifying the fluorescence emitted by the excited antibody-photosensitive substance adsorbed on the tumor cells C. Since the second detection unit 101 is rotatable about the axial center of the shaft 20, a position of a lesion extent in which the tumor cells C are present in the peripheral direction can be specified based on image information obtained by the second detection unit 101. The treatment apparatus 10 can simultaneously destroy the tumor cells C by emitting the excitation light from the light emitting unit 32 and diagnose a lesion site by detecting the fluorescence by the second detection unit 101.

When the operator determines that the fluorescence disappears by the display device 80 or a predetermined time passes, the operator can determine that the tumor cells C are sufficiently destroyed and stops emitting the excitation light. Thereafter, the operator deflates the light adjustment unit 90, and draws the treatment apparatus 10 out of the cervical canal CC and the vagina V. Accordingly, this treatment method ends.

As described above, the treatment apparatus 10 according to the third embodiment is the treatment apparatus 10 that detects and destroys the tumor cell C by irradiating the antibody-photosensitive substance adsorbed on a tumor cell membrane with the excitation light. The treatment apparatus includes: the light adjustment unit 90 configured to change the direction of the light emitted from the light irradiation unit 30; the base shaft 24 having the distal portion at which the light adjustment unit 90 is disposed; the light irradiation unit 30 configured to emit the excitation light of the antibody-photosensitive substance through the light adjustment unit 90; and the distal direction fluorescence detection unit 100 configured to detect the fluorescence emitted by the excited antibody-photosensitive substance and movable in the peripheral direction centered on an axis substantially parallel to an axial center of the base shaft 24.

According to the treatment apparatus 10 described above, since the distal direction fluorescence detection unit 100 is movable in the peripheral direction, the fluorescence emitted by the antibody-photosensitive substance excited by the excitation light from the light irradiation unit 30 can be detected by the distal direction fluorescence detection unit 100 at a plurality of positions in the peripheral direction. Therefore, according to the treatment apparatus 10, it is possible to minimally invasively specify the position of the lesion extent in the peripheral direction and check progress of treatment while performing the treatment for destroying the tumor cells C without excising a tissue. Note that, instead of the distal direction fluorescence detection unit 100, the side direction fluorescence detection unit 40 may be provided. In this case, the position of the lesion extent in the peripheral direction in the side direction Y of the light adjustment unit 90 can be specified.

The treatment method according to the third embodiment is a treatment method for detecting and destroying the tumor cell C by irradiating the antibody-photosensitive substance adsorbed on a cell membrane of a tumor developed in at least a part in a range from the cervix U to the vagina V with excitation light. The treatment method includes: administering the antibody-photosensitive substance into the body; after the administration, widening the vagina V by the vaginal speculum 200, and inserting the treatment apparatus 10 into the vagina V, the treatment apparatus 10 including the elongated base shaft 24, the light adjustment unit 90 disposed at the distal portion of the base shaft 24 and configured to change the direction of light, the light irradiation unit 30 configured to emit the excitation light of the antibody-photosensitive substance, and the distal direction fluorescence detection unit 100 configured to detect the fluorescence emitted by the excited antibody-photosensitive substance; inflating the light adjustment unit 90 in accordance with the shape of the organ; and while emitting the excitation light by the light irradiation unit 30 from an inside of the light adjustment unit 90, detecting the fluorescence emitted by the antibody-photosensitive substance by moving the distal direction fluorescence detection unit 100 in a rotation direction centered on an axis substantially parallel to the axial center of the base shaft 24, and checking disappearance or reduction of the fluorescence.

Accordingly, according to this treatment method, by emitting the excitation light from the inside of the light adjustment unit 90 disposed at the site close to the uterine vagina UV in the vagina V and detecting the fluorescence by the distal direction fluorescence detection unit 100, it is possible to minimally invasively treat the tumor distributed in the cervix U near the vagina V, specify the lesion extent, and check the progress of the treatment without excising a tissue. Note that the light irradiation unit 30 may be capable of emitting the excitation light in a direction other than the side direction Y as long as the light irradiation unit 30 is capable of emitting the excitation light in the side direction Y.

Note that the treatment apparatus 10 according to the third embodiment may be used for treatment of an organ other than the cervix U. For example, the treatment apparatus 10 can be used for treatment of a site that forms a wide space to some extent such as an esophagus and a uterine corpus.

### Fourth Embodiment

As illustrated in FIGS. 14A and 14B, the treatment apparatus 10 according to a fourth embodiment is different from that of the first embodiment in that the treatment apparatus 10 according to the fourth embodiment is provided with a puncture portion 28 at a distal end of the shaft 20. The treatment apparatus 10 is suitable for treating and diagnosing a tumor positioned at a position deep from a tissue surface.

The shaft 20 includes the puncture portion 28 at the distal end of the shaft 20 for puncturing a biological tissue. The puncture portion 28 is formed in a sharp needle shape. Note that the puncture portion 28 may not be sharp as long as the puncture portion 28 can puncture. The puncture portion 28 that is not sharp is, for example, an electrode or a laser irradiation unit. Therefore, a treatment target of the treatment apparatus 10 is not limited to the cervix U, and can be applied to all sites.

Next, a treatment method using the treatment apparatus 10 according to the fourth embodiment will be described.

First, an antibody-photosensitive substance is administered into a body, for example, by intravenous administration. After approximately 12 hours to 36 hours from the administration, while checking a distal portion of the treatment apparatus 10 by visual observation or based on an image by an image detecting device capable of detecting a tomographic image of a living body such as an ultrasonic diagnostic device, a computed tomography (CT), or a magnetic resonance imaging (MRI), an operator pierces the puncture portion 28 to the tissue surface from which the puncture portion 28 can reach the vicinity of the tumor cells C, and inserts the shaft 20 to a position where the tumor cells C are present in the side direction Y of the light transmitting portion 21 of the shaft 20.

Next, the operator disposes the light emitting unit 32 of the light irradiation unit 30 inside the distal shaft 25. A position of the light emitting unit 32 can be, for example, a most distal position where the light emitting unit 32 is reachable in a lumen of the distal shaft 25, but is not limited to the most distal position. Thereafter, the operator operates the light output device 60 to supply excitation light to the light irradiation unit 30. Accordingly, the light emitting unit 32 inside the shaft 20 can effectively emit the excitation light to the tumor cells C positioned in the cervix U. Similarly to the method described in the first embodiment, the operator can emit the excitation light from the inside of the shaft 20 inserted into the punctured tissue, detect the fluorescence by the detection unit 42, emit the excitation light from the light emitting unit 32, detect the fluorescence by the detection unit 42, move the detection unit 42, specify a range of the tumor cells C, check destruction of the tumor cells C, and stop emitting the excitation light. Similarly to the method described in the first embodiment, the operator can perform the above series of procedures (i.e., repeated procedures) once or more inside the punctured tissue.

When the operator determines that the fluorescence disappears by the display device 80 or a predetermined time passes, the operator determines that the tumor cells C are sufficiently destroyed and stops emitting the excitation light. Thereafter, the operator draws the treatment apparatus 10 out of the tissue. Accordingly, this treatment method ends.

As described above, the treatment apparatus 10 according to the fourth embodiment includes the puncture portion 28 disposed at the distal end of the shaft 20. Accordingly, even when there is a lesion at a position deep from the tissue surface, by puncturing the tissue and inserting the shaft 20 into the tissue, it is possible to treat the lesion, specify a lesion extent, and check progress of the treatment.

The treatment method according to the fourth embodiment is a treatment method for detecting and destroying the tumor cell by irradiating the antibody-photosensitive substance adsorbed on a tumor cell membrane with the excitation light. The treatment method includes: administering the antibody-photosensitive substance into the body; after the administration, forming a hole in a living body by the puncture portion 28 and inserting the distal portion of the treatment apparatus 10 into the living body while checking the distal portion of the treatment apparatus 10 by visual observation or based on the image obtained by the image detecting device such as the ultrasonic diagnostic device, CT or MRI, the treatment apparatus 10 including the elongated tubular shaft 20 having optical transparency, the puncture portion 28 disposed at the distal end of the shaft 20, the light irradiation unit 30 configured to emit the excitation light of the antibody-photosensitive substance, and the side direction fluorescence detection unit 40 configured to detect, from the side direction Y perpendicular to the axial direction X of the shaft 20, the fluorescence emitted by the excited antibody-photosensitive substance; emitting the excitation light by the light irradiation unit 30 in the side direction Y from the inside of the shaft 20; and awhile emitting the excitation light, detecting the fluorescence emitted by the antibody-photosensitive substance by moving the side direction fluorescence detection unit 40 in the axial direction X with respect to the shaft 20, and checking disappearance or reduction of the fluorescence.

Accordingly, according to this treatment method, when the lesion is at a position deep from the tissue surface, it is possible to puncture the tissue to cause the treatment apparatus to reach the vicinity of the tumor cells C, and detect the fluorescence by moving the side direction fluorescence detection unit 40 in the axial direction X while emitting the excitation light from the inside of the punctured tissue. Therefore, it is possible to specify a position of the lesion extent along the axial direction X and check the progress of the treatment while performing the treatment for destroying the tumor cells C by the excitation light.

Note that the disclosure is not limited to the embodiments described above, and various modifications can be made by those skilled in the art within a scope of the technical idea of the disclosure. Therefore, a part of a configuration included in each embodiment can be appropriately applied to other embodiments. For example, the rotatable distal direction fluorescence detection unit 100 according to the third embodiment may be applied to the second embodiment. The puncture portion 28 may be disposed at the distal end of the shaft 20 according to the first to third embodiments as in the shaft 20 according to the fourth embodiment.

The detailed description above describes embodiments of a treatment apparatus and a treatment method for cervical cancer. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A treatment apparatus that detects and destroys a tumor cell by irradiating an antibody-photosensitive substance adsorbed on a tumor cell membrane with excitation light, the treatment apparatus comprising:
an elongated tubular shaft having optical transparency;
a light irradiation unit configured to emit the excitation light of the antibody-photosensitive substance from an inside of the shaft to a side direction perpendicular to an axial direction of the shaft; and
a side direction fluorescence detection unit configured to detect, from the side direction perpendicular to the axial direction of the shaft, fluorescence emitted by the excited antibody-photosensitive substance, and the side direction fluorescence detection unit being configured to be movable in the axial direction with respect to the shaft.

2. The treatment apparatus according to claim 1, further comprising:
a puncture portion disposed at a distal end of the shaft.

3. The treatment apparatus according to claim 1, further comprising:
a light adjustment unit configured to change a direction of the excitation light emitted from the light irradiation unit;
a distal direction fluorescence detection unit configured to detect the fluorescence from a distal direction of the shaft;
the shaft including a base shaft having a distal portion at which the light adjustment unit is disposed, and a distal shaft protruding from the light adjustment unit in the distal direction and having optical transparency; and
wherein the light irradiation unit is configured to be movable between an inside of the distal shaft and an inside of the light adjustment unit.

4. The treatment apparatus according to claim 1, wherein the side direction fluorescence detection unit includes a ring-shaped scatterer disposed along a peripheral direction of the light irradiation unit and movable in the axial direction along an outer peripheral surface of the light irradiation unit, and an optical waveguide disposed on a proximal side of the scatterer.

5. The treatment apparatus according to claim 1, wherein the side direction fluorescence detection unit includes a plurality of optical fibers, and a plurality of incident portions of the optical fibers are disposed along a peripheral direction of the light irradiation unit, and are movable in the axial direction along an outer peripheral surface of the light irradiation unit.

6. The treatment apparatus according to claim 5, further comprising:
a reflector is disposed on an outer surface of the optical fiber facing an outer peripheral surface of the light irradiation unit.

7. A treatment method for detecting and destroying a tumor cell by irradiating an antibody-photosensitive substance adsorbed on a tumor cell membrane of a cervix with excitation light, the treatment method comprising:
administering the antibody-photosensitive substance into a body;
after the administration of the antibody-photosensitive substance into the body, widening a vagina by a vaginal speculum, and inserting a treatment apparatus into the vagina, the treatment apparatus including
an elongated tubular shaft having optical transparency;
a light irradiation unit configured to emit the excitation light of the antibody-photosensitive substance; and
a side direction fluorescence detection unit configured to detect, from a side direction perpendicular to an axial direction of the shaft, fluorescence emitted by the excited antibody-photosensitive substance;
inserting the shaft into a cervical canal while visually checking a distal portion of the shaft and an external uterine ostium;
emitting the excitation light by the light irradiation unit from an inside of the shaft in the side direction; and
while emitting the excitation light, detecting the fluorescence emitted by the antibody-photosensitive substance by moving the side direction fluorescence detection unit in the axial direction with respect to the shaft, and checking disappearance or reduction of the fluorescence.

8. The treatment method according to claim 7, wherein the treatment apparatus further includes a light adjustment unit disposed at a distal portion of the base shaft and configured to change a direction of light, an elongated tubular distal shaft protruding from the light adjustment unit in a distal direction and having optical transparency, and a distal direction fluorescence detection unit configured to detect the fluorescence from a distal side, the treatment method further comprising:
inserting the distal shaft into a cervical canal while visually checking the distal shaft;
inflating the light adjustment unit in accordance with a shape of an organ;
emitting the excitation light by the light irradiation unit in the side direction from an inside of the distal shaft;
while emitting the excitation light, detecting the fluorescence emitted by the antibody-photosensitive substance by moving the side direction fluorescence detection unit in the axial direction with respect to the distal shaft, and checking disappearance or reduction of the fluorescence;
moving the light irradiation unit to a position where the light adjustment unit is disposed in the axial direction; and
while emitting the excitation light through the light adjustment unit, detecting the fluorescence emitted by the antibody-photosensitive substance by the distal direction fluorescence detection unit, and checking the disappearance or reduction of the fluorescence.

9. The treatment method according to claim 7, further comprising:
after the administration of the anti-body substance into the body, forming a hole in the living body by a puncture portion and inserting a distal portion of the treatment apparatus into the living body while checking the distal portion of the treatment apparatus by visual observation or based on an image obtained by an image detecting device, the treatment apparatus including:
the elongated tubular shaft having optical transparency;
the puncture portion disposed at a distal end of the shaft;
the light irradiation unit configured to emit the excitation light of the antibody-photosensitive substance; and
the side direction fluorescence detection unit configured to detect, from the side direction perpendicular to the axial direction of the shaft, fluorescence emitted by the excited antibody-photosensitive substance.

10. The treatment method according to claim 7, further comprising:
after the checking of the disappearance or reduction of the fluorescence, the treatment method comprises:
moving the light irradiation unit in the axial direction; and
while emitting the excitation light by the light irradiation unit from the inside of the shaft in the side direction, detecting the fluorescence emitted by the antibody-photosensitive substance by moving the side direction fluorescence detection unit in the axial direction with respect to the shaft, and checking the disappearance or reduction of the fluorescence.

11. The treatment method according to claim 10, further comprising:
performing more than once the moving of the light irradiation unit in the axial direction, and the detecting of the fluorescence emitted by the antibody-photosensitive substance by moving the side direction fluorescence detection unit in the axial direction with respect to the shaft, and checking the disappearance or reduction of the fluorescence.

12. The treatment method according to claim 7, further comprising:
checking a progress of a treatment of the tumor cell without excising a tissue of the tumor cell.

13. The treatment method according to claim 7, wherein the side direction fluorescence detection unit includes a ring-shaped scatterer disposed along a peripheral direction of the light irradiation unit and movable in the axial direction along an outer peripheral surface of the light irradiation unit, and an optical waveguide disposed on a proximal side of the scatterer.

14. The treatment method according to claim 7, wherein the side direction fluorescence detection unit includes a plurality of optical fibers, and a plurality of incident portions of the optical fibers are disposed along a peripheral direction of the light irradiation unit, and are movable in the axial direction along an outer peripheral surface of the light irradiation unit.

15. The treatment method according to claim 14, further comprising:
disposing a reflector on an outer surface of the optical fiber facing an outer peripheral surface of the light irradiation unit; and
preventing the excitation light emitted from the light irradiation unit from entering the detection unit with the reflector.

16. The treatment method according to claim 8, further comprising:
after the checking of the disappearance or reduction of the fluorescence, the treatment method comprises:
moving the light irradiation unit in the axial direction; and
while emitting the excitation light by the light irradiation unit from the inside of the shaft in the side direction, detecting the fluorescence emitted by the antibody-photosensitive substance by moving the side direction fluorescence detection unit in the axial direction with respect to the shaft, and checking the disappearance or reduction of the fluorescence.

17. The treatment method according to claim 9, further comprising:
after the checking of the disappearance or reduction of the fluorescence, the treatment method comprises:
moving the light irradiation unit in the axial direction; and
while emitting the excitation light by the light irradiation unit from the inside of the shaft in the side direction, detecting the fluorescence emitted by the antibody-photosensitive substance by moving the side direction fluorescence detection unit in the axial direction with respect to the shaft, and checking the disappearance or reduction of the fluorescence.

18. A treatment method for detecting and destroying a tumor cell by irradiating an antibody-photosensitive substance adsorbed on a tumor cell membrane of a cervix with excitation light, the treatment method comprising:
administering the antibody-photosensitive substance into a living body;
widening a vagina by a vaginal speculum, and inserting a treatment apparatus into the vagina;
inserting of the treatment apparatus into a cervical canal while visually checking a distal portion of a tubular shaft of the treatment apparatus and an external uterine ostium;
emitting an excitation light from the treatment apparatus from an inside of the tubular shaft in a side direction; and
detecting fluorescence emitted by the antibody-photosensitive substance by moving the treatment apparatus in the axial direction with respect to the tubular shaft, and checking disappearance or reduction of the fluorescence.

19. The treatment method according to claim 18, further comprising:
inserting the treatment apparatus into a cervical canal;
inflating a light adjustment unit in accordance with a shape of an organ;
emitting the excitation light from treatment apparatus from an inside of a distal shaft of the treatment apparatus;
detecting the fluorescence emitted by the antibody-photosensitive substance by moving the treatment apparatus in the axial direction with respect to the distal shaft of the treatment apparatus, and checking disappearance or reduction of the fluorescence;
moving the treatment apparatus to a position where the treatment apparatus is disposed in the axial direction; and
detecting the fluorescence emitted by the antibody-photosensitive substance by the treatment apparatus, and the checking the disappearance or reduction of the fluorescence.

20. The treatment method according to claim 18, further comprising:
forming a hole in the living body by a puncture portion of the treatment apparatus and inserting a distal portion of the treatment apparatus into the living body while checking the distal portion of the treatment apparatus by visual observation or based on an image obtained by an image detecting device.
